(19) United States
(12) Patent Application Publication
(10) Pub. No.: US 2011/0144626 A1
(43) Pub. Date: Jun. 16, 2011

(54) MEDICAL FLUID COMMUNICATION COUPLINGS

(76) Inventor: Judith Hall, Cardiff (GB)

(21) Appl. No.: 13/054,285

(22) PCT Filed: Jul. 14, 2009
(86) PCT No.: PCT/GB2009/050851

§ 371 Date: Feb. 11, 2011

(30) Foreign Application Priority Data
Jul. 14, 2008 (GB) 0812837.3

(51) Int. Cl. A61M 39/10

(52) U.S. Cl. 604/535

(57) ABSTRACT

A medical coupling system is disclosed for coupling medical apparatus in fluid communication, the system comprising a plurality of groups of couplings, each member (10) of each group including first (32) and second (22) complementary connector parts, each first or second connector part (32/22) of a coupling being shaped to be mateable only together with corresponding connector parts in the same group, one end of the first connector part of each coupling having a shaped spigot (36), adapted for non-rotatable fitting into an aperture (26) in the corresponding second connector part (22), the first connector part further including a sleeve (40) rotatably mounted thereon, having an internal screw thread (43) for engaging with a complementary external thread (23) of the second connector part for holding the first and second connector parts together in use. The outer peripheral shapes of flanges (38/28) on the connector parts, and/or the sleeve (40) may be enlarged versions of the shape of the spigot for ready recognition that the two halves are mateable and for easy turning.

This invention relates to medical couplings for allowing fluid communication, for example, to and from patients receiving medical treatment or therapy. In particular, but not exclusively, the invention relates to the provision of groups of couplings each coupling being formed from a pair of connectors, each connector in the pair being mateable only with corresponding connectors in the group bit not with other connectors of a different group, so that the wrong connectors cannot be put together.

Typically, fluid communication with patients is via plastic tubing or the like which interconnects with medical devices. Such communication allows the introduction of fluids, drugs or gasses into patients via various routes, or the removable of fluids from a patient. Usually, in an operating theatre, several tubes can be used at any one time for, all for differing purposes. Often, these tubes are twisted together or disappear under patient covers and so it is quite possible that mistakes in identification of various tubes can be made. As an example of what can go wrong, injecting the wrong fluid into a patient can have fatal consequences.

Whilst uniquely shaped connectors which will fit together but do not fit with other connectors have been proposed, the inventor has realised that what is needed is a range of couplings and a range of couplings which make obvious which connector pairs will fit together.

According to a first aspect the invention provides a medical coupling system for coupling medical apparatus in fluid communication, the system comprising a plurality of groups of couplings, each member of each group including first and second complementary connector parts, each first or second connector part of a coupling being shaped to be mateable only together with a corresponding connector parts in the same group, one end of the first connector part of each coupling having a shaped spigot, adapted for non-rotatable fitting into an aperture in the corresponding second connector part, one of the first or second connector parts further including a sleeve rotatably mounted or mountable thereon, having a screw thread for engaging with a complementary thread of a respective connector part for holding the first and second connector parts together in use.

Preferably, said aperture has an internal surface corresponding to the shape of the spigot, the shape of the spigot being polygonal or other than circular.

Preferably, the sleeve is captively held to its connector part such that securing or release of the two connector parts is accomplished by rotation of the sleeve relative to the first and second parts.

More preferably, the spigot protrudes beyond the sleeve such that it is engageable with the aperture of the corresponding connector part before the engagement of said threads.

In an embodiment, the outer peripheral shape of the sleeve is substantially an enlarged version of the outer peripheral shape of the spigot.

Conveniently, one or both connector parts may include a flange of increased size in comparison to the remainder of the part, said flange(s) having an outer peripheral shape substantially the same as the size and shape of the outer peripheral shape of the sleeve.

Additionally or alternatively, one or both connector parts may include a flange of increased size in comparison to the remainder of the part, said flange(s) having an outer peripheral shape which is substantially an enlarged version of the outer shape of the spigot. According to a second aspect of the invention there is provided a medical fluid coupling allowing fluid communication of medical apparatus with a patient, the coupling comprising two connector parts connectable to provide said fluid communication, a first connector part comprising a spigot having an outer peripheral shape and a second connector part including an aperture for non-rotatably receiving the spigot, the aperture having a shape complimentary to the outer shape of the spigot, the two connector parts being releasably securable by means of a threaded sleeve rotatably mounted or mountable to one of the connector parts and engageable with complementary threads associated with the other of the parts, the coupling being characterised in that either: the sleeve includes an outer peripheral shape which is an enlarged version of the shape of the spigot; or the first and second parts each have a flange larger in size in comparison with the remaining first and second parts, each said flange having an outer peripheral shape which is an enlarged version of the shape of the spigot; or both the sleeve and the flanges are shaped to be the same enlarged version of the spigot shape.

In this way the shape of the spigot can be easily identified by simply viewing the sleeve and/or flange shape, which is especially useful if the coupling is within say a sterile package.

In an embodiment, said sleeve is mounted on the first part and substantially surrounds the spigot, the second part including also a threaded portion for being captured by a complementary threaded portion of the sleeve for holding the spigot within the aperture.

A coupling system may be employed which utilises the couplings of the second aspect.

The coupling of the second aspect may include the features of the couplings described in relation to the first aspect.

The invention extends to any novel feature or novel combination of features described or illustrated herein, whether or not that combination is explicitly described or illustrated.

Embodiments of the invention will now be described below, by way of example only, with reference to the drawings wherein,

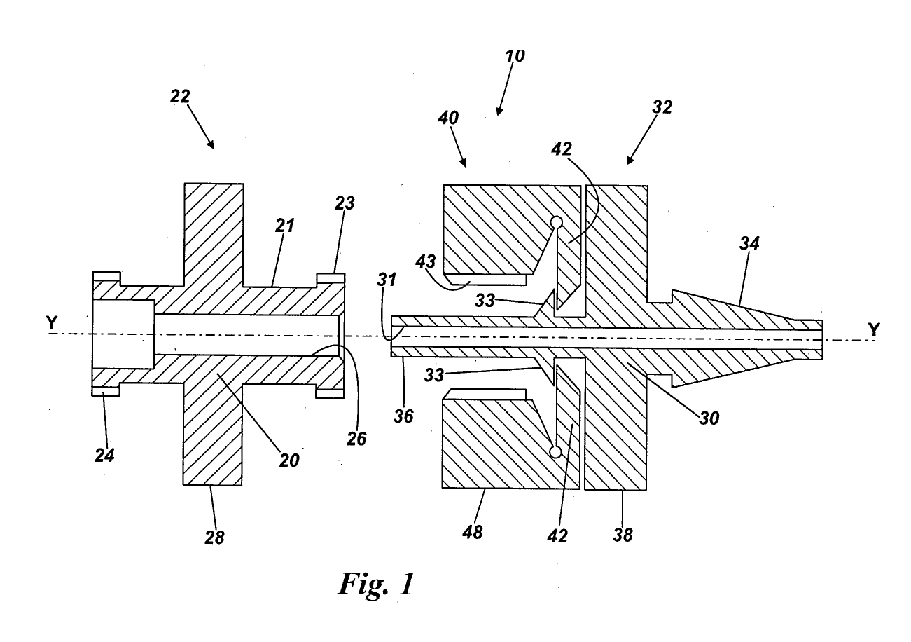

FIG. 1 shows a section of a coupling according to the invention;

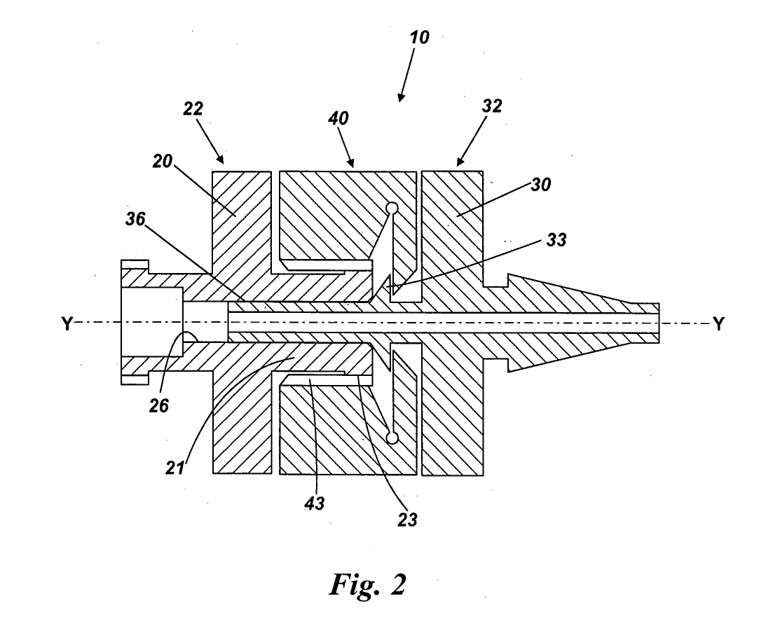

FIG. 2 shows the coupling illustrated in FIG. 1 in an assembled condition;

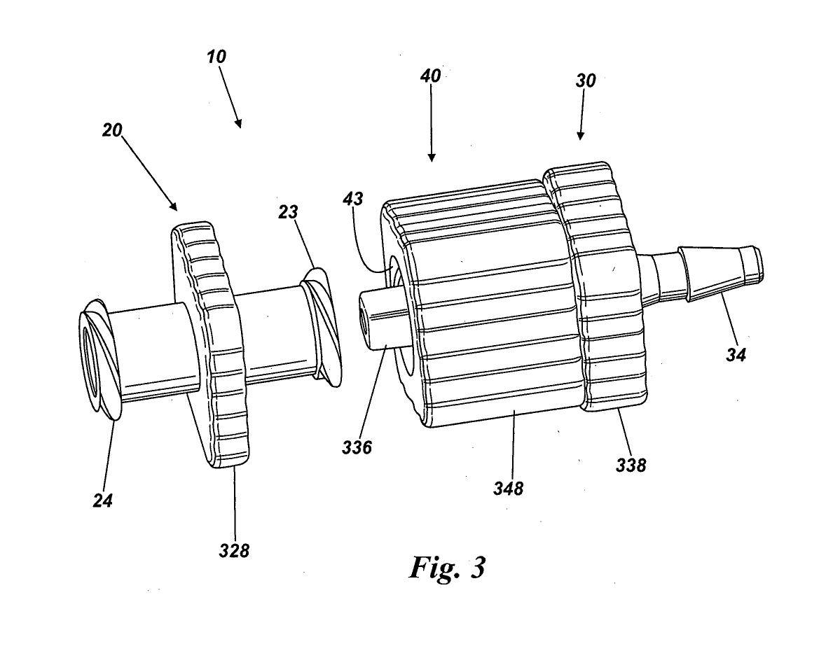

FIGS. 3, 4 and 5 show differently shaped couplings useable in a range of couplings; and

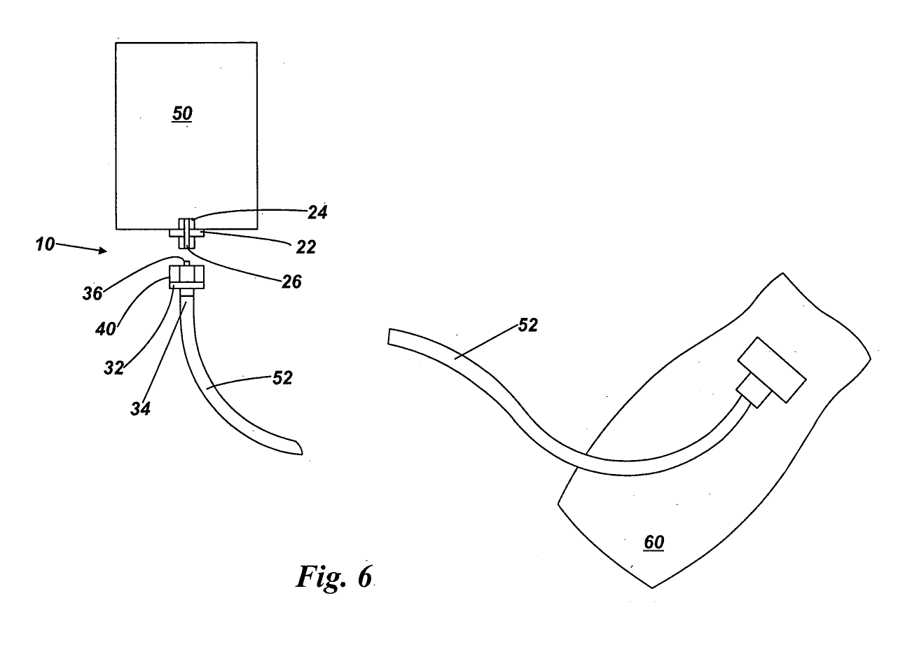

FIG. 6 shows a coupling in use in a typical application.

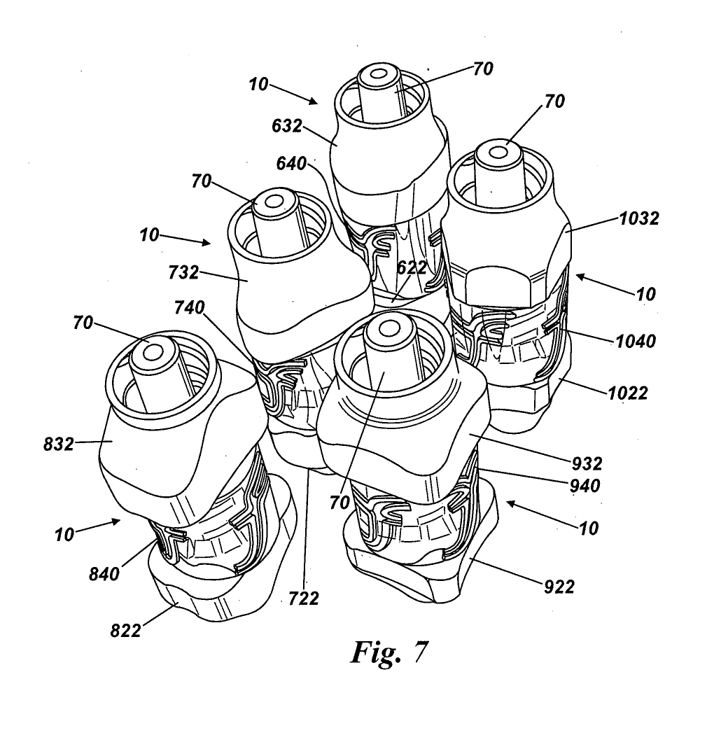

FIG. 7 shows a group of further couplings shown assembled.

Referring to FIG. 1, there is shown generally the elements of a coupling 10 in an un-coupled condition. The coupling 10 includes a pair of connectors 22 and 32 each of which includes a body parts 20 and 30 respectively, centred generally about an axis Y.

Connector body 20 includes an adapter portion 24 suitable for generally permanent connection to a piece of medical apparatus such as a syringe, intravenous fluid reservoir, valve, or the like. In this illustration the portion has a thread, although, any means suitable for generally permanent connection could be used, for example an integral moulding with the medical apparatus could be employed. The connector body 20 includes also an aperture 26 which will be described in more detail below. The connector body 20 further includes a flanged portion 28 and a portion 21 surrounding the aperture 26 which includes a multi-start male thread 23, again explained in more detail below.

The connector body 30 also includes an adapter portion 34 for permanently connecting to further medical apparatus such as a supply tube or the like. Although a cone shaped adapter 34 has been illustrated, it will be appreciated that any means of permanently connecting the connector body 30 to the further medical apparatus could be employed; again, the connector body 30 could be made integral with the further medical apparatus. Connector body 30 includes a flanged portion 38 and a spigot 36, the latter being adapted to be non-rotatably received in the aperture 26 of the connector body 20. The connector body 30 includes a generally central through hole 31 which allows fluid communication through the coupling 10 when the connectors 20 and 30 are fitted together.

A sleeve 40 is captively held to the connector body 30 by means of fingers 42 which are resiliently sprung over a raised portion 33 of the spigot 36 during assembly. Sleeve 40 is rotatable relative to the connector body 30 generally about the axis Y. The sleeve 40 includes an internal thread 43 which is a multi start thread and is complementary to the male thread 23 on the protrusion 21 of the connector body 20.

Referring additionally to FIG. 2, the coupling is shown in the assembled condition in that Figure. In use, spigot 36 is offered into the aperture 26. The spigot and aperture are constructed to provide a complementary sliding fit, and, as will be described in more detail below, may be of a polygonal or non-circular shape such that the two parts cannot rotate relatively. As the spigot 36 and aperture 26 are offered further together the complementary threads 23 and 43 come into engagement and the sleeve 40 can be rotated relative to the now mated connector bodies 20 and 30. Rotation of the sleeve 40 relative to the connector bodies 20 and 30 draws the spigot 36 further into the aperture 26 until the raised portion 33 abuts the end of the portion 21 closest to the connector body 30.

Release of the coupling is carried out by reversing the coupling actions mentioned above.

Referring to FIGS. 3, 4 and 5, different embodiments of the coupling 10 illustrated generally in FIGS. 1 and 2 are shown, in an uncoupled state. In practice, the embodiments shown in FIGS. 3, 4 and 5 represent three examples of groups of couplings that would be used in a system comprising a plurality of coupling groups wherein only connectors in the same group can be connected together, to avoid mistakes during treatment caused by connecting the wrong pieces of medical apparatus together. However, the basic construction for each coupling is the same as illustrated in FIGS. 1 and 2, and therefore the cost of manufacturing each coupling is reduced because each coupling has common features.

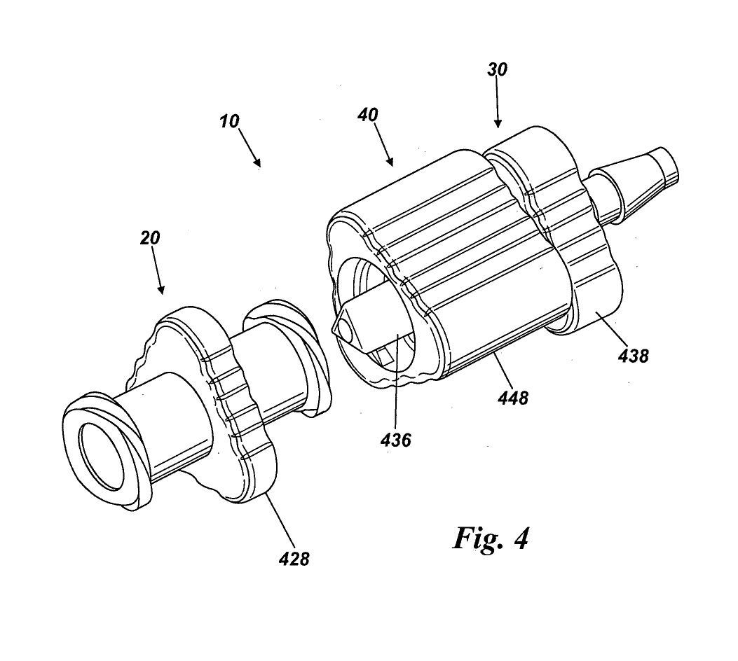

Referring specifically to FIG. 3, there is shown a coupling 10 having connector bodies 20 and 30 as described above and a sleeve 40 also described as above. The coupling includes portions 24 and 34 adapted to fit other medical apparatus as described above. In this embodiment there is provided a square shaped spigot 336 for fitting in a non-rotatable manner into a complementary shaped aperture (not shown) in connector body 20. Male thread 23 and female thread 43 are engaged as described above in order to bring the connector bodies 20 and 30 fully together. It will be noted that the flanged portions 328 and 338 have outer peripheries which are enlarged versions of the shape of the spigot 336 i.e. the flanged portions too are square. Additionally, the sleeve 40 has an outer peripheral shape 348 which too is of the same square shape as the flanges 328 and 338. The purpose of the consistency in the geometry of the shape of the flanges, the sleeves and the spigots is to provide easy user identification of complementary connector parts during use. Referring specifically to FIG. 4, there is shown a further coupling 10 which is similar in construction to the couplings described above. However, this coupling includes a triangular spigot 436 for being received in a complementary aperture (not shown) in connector body 20. It will be noted that the flanges 428 and 438 of the connector bodies 20 and 30 also have a triangular outer shape and the sleeve 40 also includes a triangular outer shape 448.

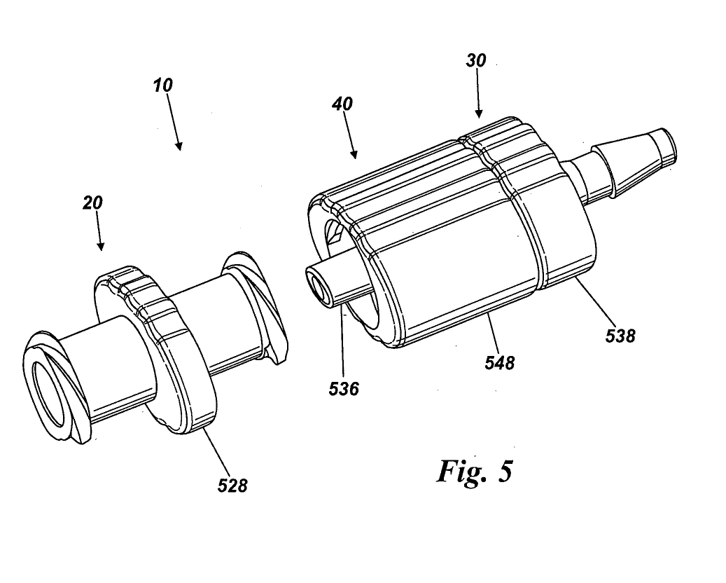

Referring to FIG. 5, an oval spigot 536 is illustrated for being received in a complementary oval shaped aperture (not shown) in connector body 20. In keeping with the embodiments of the coupling shown in FIGS. 3 and 4, the coupling shown in FIG. 5 includes flanges 528 and 538 which are oval in shape and a sleeve 40 having an outer periphery 548 which is also oval.

In order to reduce manufacturing costs the sleeve 40 can be made one shape, for example cylindrical, so that the same sleeve can be used for each of the connectors shown in FIGS. 3, 4 and 5.

FIG. 6 illustrates a typical application of the coupling 10. In this illustration, an intravenous fluid supply reservoir 50 is integrally connected to the connector 22 via a connecting portion 24. The connector body 22 includes an aperture 26. Connector body 32 includes a spigot 36 which will only fit into aperture 26 so that only intravenous reservoirs can be connected to the connector body 32 and only intravenous supply lines 52 will have the body 32 irremovably fitted thereto. Connection of the coupling 10 is as described above and following coupling the safe intravenous supply to a patient 60 is accomplished, without the risk of connecting the wrong medical apparatus together. In the same vicinity, oxygen supplies or other medical equipment will carry a connector from a different group and so it will be impossible to connect the intravenous feed 52 to the wrong apparatus. Using a range of couplings only certain pairs of which can be connected together, allows a safer means of ensuring that the correct fluid is given to or taken from the patient. In particular, use of a shaped spigot 36 and an aide memoire in the form of a similarly shaped sleeve and/or flanges on the connector bodies allows easy identification of the connector pairs that will fit together.

Shown in FIG. 7 is a group of five couplings 10 which have pairs of parts that can only be connected together and cannot be connected wrongly to other parts. The couplings have a construction similar to the construction of the couplings described above. Connector bodies 632, 732, 832, 932 and 1032 are, in this instance, each formed with a tube holding formation 70 which has an internally threaded outer collar and an inner boss. Each of the connector bodies 632, 732, 832, 932 and 1032 have a flange portion having a different outer peripheral shape, to match the shape of a respective spigot (not shown) as described above. Similarly shaped further connector bodies 622, 722, 822, 922 and 1022 are paired with their matching connector bodies 632, 732, 832, 932 and 1032 respectively. In this embodiment, each coupling shares a commonly shaped sleeve 640, 740, 840, 940 and 1040. These sleeves are transparent, although they could also be translucent. This transparency or translucency allows a user to view the spigot more clearly.

In all the embodiments, the shape of the flanges of the connector bodies, and the shape of their spigots is the same, and when they are aligned, such that the flanges are in the same rotational position relative to the axis of the coupling, then the spigot and its respective aperture 26 will be aligned also, to come together without the need to twist one body relative to the other.

Although certain embodiments of the invention have been described above, it will be apparent to the skilled addressee that various modifications, alternatives and variants could be employed without departing from the ambit of the invention. For example, oval, triangular and square spigots 36 have been illustrated but it will be apparent that other shapes could be employed, for example other regular or non-regular polygonal shapes could be employed such as pentagons, heptagons or octagons. Also, other shapes having non-linear sides could be employed such as quadrants, crescent shapes or combinations of regular or irregular shapes. As a means of easy identification the shape of the spigot 36, in addition to the corresponding shape of the flanges and sleeve, the coupling parts could be correspondingly coloured.

Although it is preferred that the coupling is manufactured from moulded plastics, the coupling parts could be made from other materials such as metals, ceramics, natural materials or combinations of these. It is envisaged that the threads 23 and 43 will be the same for each connector pair however, as a further enhancement, it would be possible to provide different threads for each of the connector pairs in different groups to further avoid the risk of connecting the wrong medical apparatus together.

The couplings described and illustrated could be used for fluid or gas communication and could be used, for example in intravenous, arterial, epidural, gastric or air supplying applications.

It would be possible not only to provide a group of connectors differentiated by their general shape, but also connectors having the same general geometry but different sizes to allow greater or lesser flow rates. It is envisaged that similarly shaped connectors would be employed in transferring the same fluids, for example square shaped connectors should supply only fluids intravenously, so, even if it were possible to inadvertently connect differently sized connector pairs having otherwise similar geometry, that should in practice be unproblematic. However, for good practice this should be avoided and therefore, differently sized connectors could be constructed such that the smaller sized version of the pair would simply not fit into, or fall out of larger connector, if connection were attempted. Thus connectors, as described above, could be provided having a range of sizes corresponding to standard gauge sizes 6 to 32.

1. A medical coupling system for coupling medical apparatus in fluid communication, the system comprising a plurality of groups of couplings, each member of each group including first and second complementary connector parts, each first or second connector part of a coupling being shaped to be mateable only together with corresponding connector parts in the same group, one end of the first connector part of each coupling having an other than circular shaped spigot, adapted for non-rotatable fitting into an aperture in the corresponding second connector part, one of the first or second connector parts further including a sleeve rotatably mounted or mountable thereon, having a screw thread for engaging with a complementary thread of a respective connector part for holding the first and second connector parts together in use, and wherein one or both connector parts include a flange of increased size in comparison to the remainder of the part, said flange(s) having an outer peripheral shape which is substantially an enlarged version of the other than circular outer shape of the spigot.

2. A medical coupling system as claimed in claim 1, wherein said aperture has an internal surface corresponding to the shape of the spigot, the shape of the spigot being polygonal or other than circular.

3. A medical coupling system as claimed in claim 1 wherein, the sleeve is captively held to its connector part and the securing or release of the two connector parts is accomplished by rotation of the sleeve relative to the first and second parts.

4. A medical coupling system as claimed in claim 1 wherein, the sleeve is mounted on the first connector part and the spigot of the first connector part protrudes beyond the sleeve such that it is engageable with the aperture of the second connector part before the engagement of said threads.

5. A medical coupling system as claimed in claim 1 wherein, the outer peripheral shape of the sleeve is substantially an enlarged version of the outer peripheral shape of the spigot.

6. (canceled)

7. (canceled)

8. A medical coupling system as claimed in claim 1 wherein the sleeve is transparent or translucent.

9. A medical fluid coupling allowing fluid communication, the coupling comprising two connector parts connectable to provide said fluid communication, a first connector part comprising a spigot having an other than circular outer peripheral shape and a second connector part including an aperture for non-rotatably receiving the spigot, the aperture having a shape complimentary to the outer shape of the spigot, the two connector parts being releasably securable by means of a threaded sleeve rotatably mounted or mountable to one of the connector parts and engageable with complementary threads associated with the other of the parts, the coupling being characterised in that the first and second parts each have a flange larger in size in comparison with the remaining features of the first and second parts, each said flange having an outer peripheral shape which is an enlarged version of the other than circular shape of the spigot.

10. A medical fluid coupling as claimed in claim 9 wherein said sleeve is mounted on the first part and substantially surrounds the spigot, the second part including also a threaded portion for being captured by a complementary threaded portion of the sleeve for holding the spigot within the aperture.

11. A medical coupling system incorporating a plurality of groups of medical fluid couplings, each group comprising medical fluid couplings as claimed in claim 9 wherein the spigot shape for each group is different and will not fit into an aperture of a connector part of another group.
